(19) United States
(12) Patent Application Publication
(10) Pub. No.: US 2022/0280499 A1
(43) Pub. Date: Sep. 8, 2022

(54) NOVEL DRUG COMBINATIONS FOR TREATMENT OF A CARCINOMA

(71) Applicant: Stichting Het Nederlands Kanker Instituue-Antoni van Leeuwenhoek Ziekenhuis, Amsterdam (NL)

(72) Inventors: Rene BERNARDS, Amsterdam (NL); Haojie JIN, Amsterdam (NL)

(21) Appl. No.: 17/770,399

(22) PCT Filed: Oct. 21, 2020
(86) PCT No.: PCT/NL2020/050646

§ 371 Date: Apr. 20, 2022

(30) Foreign Application Priority Data
Oct. 21, 2019 (EP) 19204376.8

(51) Int. Cl. A61K 31/47; A61K 31/5377; A61K 31/517; A61K 31/506; A61K 31/4706; A61K 39/395; A61P 35/00

(52) U.S. Cl. CPC A61K 31/47; A61K 31/5377; A61K 31/517; A61K 31/506; A61K 31/4706; A61K 39/3955; A61P 35/00

(57) ABSTRACT

The invention relates to a method of treating a patient suffering from a carcinoma, especially a hepatocellular carcinoma. The methods of the invention employ a specific combination of two protein kinase inhibitors, comprising lenvatinib and an Epidermal Growth Factor Receptor (EGFR) inhibitor. The invention further relates to a pharmaceutical preparation comprising lenvatinib and an EGFR inhibitor, a kit of parts, comprising lenvatinib and an EGFR inhibitor, and to the use of the pharmaceutical preparation or the kit of parts in a method of treating a patient suffering from a hepatocellular carcinoma.

### FIELD

The invention relates to methods of treating a carcinoma based on a combination of receptor tyrosine kinase inhibitors.

### 1 INTRODUCTION

Cancer is a leading cause of death worldwide, accounting for an estimated total of 9.6 million deaths in 2018. The most common cancers are lung cancer, breast cancer and colorectal cancers, while lung cancer, colorectal cancer and stomach cancer are the most common causes of cancer death (The Global Cancer Observatory, 2019. Factsheet on cancers/39).

Hepatocellular carcinoma (HCC) is the fifth most common tumor worldwide, and the most common type of primary liver cancer. HCC occurs most often in people with chronic liver diseases, such as cirrhosis caused by hepatitis B or hepatitis C infection. Multiple treatment options are available for HCC including curative resection, liver transplantation, radiofrequency ablation, trans-arterial chemoembolization, radioembolization and the administration of systemic multiple receptor tyrosine kinase inhibitors such as sorafenib.

The five-year survival rate of patients after curative resection of HCC has been reported to be 30 to 50%. Local ablative therapies such as radiofrequency ablation is considered a standard of care for the patients with very early and early stage cancers, resulting in 5-year survival rates ranging from 40% to 70% (Raza and Sood, 2014. World J Gastroenterol 20: 4115-4127). Trans-arterial chemoembolization (TACE) is becoming a standard treatment for the patients with intermediate-stage HCC, especially when employing embolic microspheres. TACE has been reported to achieve a partial response in 15%-62% patients, with significantly delayed tumor progression and improvement in median survival from 16-20 months (Llovet and Bruix, 2003. Hepatology 37: 429-42).

Due to the rapid progress and delayed diagnosis, the majority of liver cancer patients are diagnosed at an advanced stage. For advanced HCC patients, sorafenib (Nexavar) was the only drug approved by the US Food and Drug Administration (FDA) for treatment before 2018. It is an orally active anti-angiogenic multi-kinase inhibitor. Besides suffering from adverse side-effects and high costs, sorafenib only improves survival for less than 3 months compared to placebo in clinical trials (Llovet et al., 2008. N Engl J Med 359: 378-390; Cheng et al., 2009. Lancet Oncology 10: 25-34). Combined treatment of sorafenib and Epidermal Growth Factor Receptor inhibitors such as erlotinib showed synergistic antitumor effects in human colorectal and lung cancer cells (Martinelli et al., 2010. Clin Cancer Res 16: 4990-5001), but did not improve survival of patients with advanced HCC (Zhu et al., 2015. J Clin Oncol 33: 559-566).

Another inhibitor of multiple receptor tyrosine kinases, lenvatinib, demonstrated a non-inferior overall survival compared to sorafenib in first-line treatment of advanced HCC (13.6 vs 12.3 months), becoming a valid alternative option in the therapeutic repertoire of this disease (Kudo et al., 2018. Lancet 391: 1163-1173). Although lenvatinib showed improved clinical activity, it is still far from meeting clinical needs

There is thus a need to improve response rates and overall survival of advanced carcinoma patients, such as advanced HCC patients, by targeting molecular agents such as receptor tyrosine kinase inhibitors.

### 2 BRIEF DESCRIPTION OF THE INVENTION

To find potential drug targets that can act synergistically with lenvatinib in a carcinoma such as hepatocellular carcinoma (HCC), a synthetic lethality screen with lenvatinib was performed using CRISPR-Cas9 genetic screen targeting the human kinome in the HCC cell line SNU449. The results showed that suppression of EGFR in combination with lenvatinib caused a marked inhibition of proliferation in SNU449 cells.

This effect is surprising as previous studies had shown that an orally active inhibitor of EGFR tyrosine kinase, erlotinib, showed no improvement of sorafenib efficacy upon combination with erlotinib (Zhu et al., 2015. J Clin Oncol 33: 559-566). The lack of synergistic or additive effect of the combination of erlotinib and sorafenib suggested that EGF signaling may not be pivotal in advanced HCC (Gao et al., 2015. World J Gastroenterol 21: 12059-12070).

The invention therefore provides a combination of lenvatinib and an Epidermal Growth Factor Receptor (EGFR) inhibitor for use in a method of treating a patient in need thereof suffering from a hepatocellular carcinoma. It was surprisingly found that hepatocellular carcinoma cells reacted synergistically to treatment with a combination of lenvatinib and an EGFR inhibitor, while other cells such as lung cancer cells, pancreatic cancer cells, and colon cancer cells, did not.

Said EGFR inhibitor preferably is a EGFR tyrosine kinase inhibitor (TKI), or a binding molecule that prevents binding of a ligand to the EGFR. Said EGFR inhibitor preferably is or comprises gefitinib, erlotinib, lapatinib, cetuximab, neratinib, osimertinib, panitumumab, vandetanib, necitumumab, dacomitinib, or a combination thereof. Said patient suffering from a carcinoma may previously have been treated with an inhibitor of multiple receptor tyrosine kinases such as lenvatinib and/or sorafenib, and may have become resistant to said one or more inhibitors of multiple receptor tyrosine kinases.

Said hepatocellular carcinoma preferably has high levels of expression of EGFR. EGFR expression levels preferably are determined with a H-score method, whereby a score of 0-300 is assigned to a patient, based on the percentage of cells stained at different intensities and viewed at various magnifications (Mazieresa et al., 2013. Lung Cancer 82: 231-237). A high level of EGFR as determined with the H-score method preferably has a score of ≥200. A low level of EGFR expression as determined with the H-score method preferably has a score of <200.

The method of treating of a combination for use according to the invention preferably is combined or alternated with immunotherapy, and is preferably combined or alternated with a PD1/PDL1 inhibitor.

The invention further provides a pharmaceutical preparation, comprising lenvatinib and an Epidermal Growth Factor Receptor (EGFR) inhibitor. Said pharmaceutical preparation may comprise a pharmaceutical preparation comprising lenvatinib and a pharmaceutical preparation comprising an EGFR inhibitor.

The invention further provides a pharmaceutical preparation for use in a method of treating a hepatocellular carcinoma.

The invention further provides a kit of parts, comprising lenvatinib and an EGFR inhibitor as a combined preparation for simultaneous, separate or sequential use in the treatment of a hepatocellular carcinoma in a subject.

The invention further provides a method of treating a hepatocellular carcinoma in a subject, the method comprising the simultaneous, separate or sequential administering to the subject of lenvatinib and an EGFR inhibitor. Said method of treating preferably further comprises providing an immune checkpoint inhibitor, preferably a PD1/PDL1 inhibitor.

### 3 FIGURE LEGENDS

FIG. 1. A synthetic lethal screen identifies that EGFR inhibition confers sensitivity to lenvatinib in liver cancer cells. (a) Liver cancer cell lines were treated with increasing concentrations of lenvatinib for about two weeks. Cell growth was assessed by a colony formation assay. (b) Liver cancer cell lines were treated with increasing concentrations of lenvatinib for about 4 days. Viability was assessed by CellTiter-Blue assays. (c) The level of EGFR knockdown was determined by western blot for three independent shRNAs. HSP90 protein level served as a loading control. (d) Three independent shRNAs targeting EGFR enhance response to lenvatinib in SNU449 cells. These cells were culture with or without increasing concentration of lenvatinib for about 10 days.

FIG. 2. Combination of lenvatinib and EGFR inhibitors achieved strong synergistic effect in EGFRhigh liver cancer cell lines in vitro. High levels of EGFR expression in liver cancer cell lines correlate with synergy of the combination. Liver cancer cell lines with low or high level of EGFR were treated with increasing concentrations of lenvatinib and EGFR inhibitors (gefitinib or erlotinib), and cell growth was determined using IncuCyte assays.

FIG. 3. Biochemical interaction between lenvatinib and EGFR inhibition in liver cancer. Resistance to lenvatinib treatment in liver cancer cells is mediated through feedback activation of EGFR. Biochemical responses of liver cancer cells treated with lenvatinib, gefitinib, erlotinib or their combinations, were documented by western blot analysis. Activation of EGFR and ERK1/2 was analyzed. HSP90 served as a control.

FIG. 4. EGFR inhibitor and lenvatinib synergize to suppress liver cancer growth in a xenograft model. (a,b) Combination of gefitinib and lenvatinib suppressed tumor growth in SNU449 xenograft model. SNU449 cells were grown as tumor xenografts in nude mice. After tumor establishment (˜200 mm³) mice were treated with vehicle, lenvatinib (4 mg/kg), gefitinib (80 mg/kg), or lenvatinib (4 mg/kg) plus gefitinib (80 mg/kg), for 27 days. (a) Mean tumor volumes±standard error of the mean are shown (n=6 mice per group). (b) Mean body weight±standard error of the mean are shown (n=6 mice per group).

FIG. 5. EGFR expression levels in human tumors, as determined by immunohistochemical staining. The H-score method assigned a score of 0-300 to each patient, based on the percentage of cells stained at different intensities viewed at various magnifications. The discriminatory threshold was set at 200. All samples were classed as low (H-score<200) or high (≥200) EGFR expression.

FIG. 6. No response was obtained to a combination of lenvatinib and EGFR inhibitor gefitinib in several lung cancer cells (A), pancreatic cancer cells (B), and colon cancer cells (C) in vitro as indicated. Cancer cells were treated with lenvatinib, EGFR inhibitor gefitinib, or their combination at the indicated concentrations. The cells were fixed and stained after 5-10 days.

### 4 DETAILED DESCRIPTION

### 4.1 Definitions

The term “lenvatinib”, as is used herein, refers to the compound 4-[3-chloro-4-(cyclopropylcarbamoylamino)phenoxy]-7-methoxyquinoline-6-carboxamide, or a derivative and/or pharmaceutically acceptable salt thereof. Said salt preferably is lenvatinib mesylate. Lenvatinib is a synthetic, orally available inhibitor of especially vascular endothelial growth factor receptor 2 (VEGFR2) tyrosine kinase with potential antineoplastic activity. Lenvatinib inhibits the three main vascular endothelial growth factor receptors VEGFR1, 2 and 3, as well as fibroblast growth factor receptors (FGFR) 1, 2, 3 and 4, platelet-derived growth factor receptor (PDGFR) alpha, c-Kit, and the RET proto-oncogene, which are all tyrosine kinases.

The term “sorafenib”, as is used herein, refers to the compound 4-[4-[[4-chloro-3-(trifluoromethyl)phenyl]carbamoylamino]phenoxy]-N-methylpyridine-2-carboxamide, or a derivative and/or pharmaceutically acceptable salt thereof. Said salt preferably is sorafenib tosylate. Sorafenib inhibits the three main vascular endothelial growth factor receptors VEGFR1, 2 and 3, as well as PDGFR and the serine/threonine Rapidly Accelerated Fibrosarcoma (RAF) kinases.

The term “Epidermal Growth Factor Receptor (EGFR) inhibitor”, as is used herein, refers to an inhibitor of EGFR-signaling. EGF is a key growth factor regulating cell survival through binding to cell surface receptors. Activation of these cell surface EGF receptors by binding of a ligand such as EGF stimulates signal transduction pathways that ultimately result in blockage of the apoptotic pathway. An EGFR inhibitor either effectively hampers or inhibits binding of a ligand such as EGF to its receptor, or hampers or inhibits activation of EGFR, for example by inhibiting the tyrosine kinase activity of the receptor. A preferred EGFR inhibitor is selective for EGFR, when compared to other receptor tyrosine kinases, meaning that the molecule is at least two times more potent, preferably at least five times more potent, in inhibiting EGFR, when compared to other receptor tyrosine kinases.

The term “epidermal growth factor receptor family”, as is used herein, refers to a family of related receptors that act as receptors for members of the epidermal growth factor family (EGF family) of extracellular protein ligands. Said family comprises of four closely related receptor tyrosine kinases, termed HER1 (EGFR, ERBB1), HER2 (NEU, ERBB2), HER3 (ERBB3), and HER4 (ERBB4). A preferred receptor in the context of this invention is HER1 (EGFR, ERBB1). Said receptors can form homodimers and heterodimers, such HER1/HER2.

The term “EGFR”, as is used herein, refers to a transmembrane glycoprotein that is a member of the protein kinase superfamily. This protein is a receptor for members of the epidermal growth factor family. EGFR is a cell surface protein that binds to epidermal growth factor. Binding of the protein to a ligand induces receptor dimerization and tyrosine autophosphorylation and leads to cell proliferation. The gene is characterized by HGNC accession number 3236, Entrez Gene accession number 1956 and Ensembl accession number ENSG00000146648. The encoded protein is characterized by UniProt accession number P00533.

The term “fibroblast growth factor (FGF)”, as is used herein, refers to a family of cell signaling molecules that regulate a broad spectrum of biological functions, including cellular proliferation, survival, migration, and differentiation. The human FGF family presently consists of 22 structurally related members. FGFs can bind and activate FGF receptors, which are transmembrane glycoproteins that are members of the protein kinase superfamily. Presently, there are 4 FGF receptors known, termed FGFR1-4.

As is used herein, the term “combination” refers to the administration of effective amounts of lenvatinib and an EGFR inhibitor to a patient in need thereof. Lenvatinib and an EGFR inhibitor may be provided in one pharmaceutical preparation, or as two distinct pharmaceutical preparations.

The term “effective amount”, as used herein, means an amount of a pharmaceutical compound, such as lenvatinib and an EGFR inhibitor, that produces an effect on the carcinoma to be treated.

The term “carcinoma”, as is used herein, refers to a cancer that has an epithelial origin.

The term “immune checkpoint inhibitor”, as is used herein, refers to a molecule that blocks an inhibitory interaction between immune cells and other cells or cytokines and which may thereby increase the killing of cancer cells. Examples of checkpoint interacting molecules are PD-1/PD-L1 and CTLA-4/B7-1/B7-2. A preferred immune checkpoint inhibitor is a molecule that blocks an interaction between PD-1 and PD-L1. Said molecule that blocks an interaction between PD-1 and PD-L1 preferably is an antibody against PD1 and/or an antibody against PDL1.

### 4.2 Methods of Treatment

Provided herein is a method of treating an individual suffering from a hepatocellular carcinoma, comprising providing said individual with lenvatinib and an EGFR inhibitor.

Further provided is a use of an EGFR inhibitor in the preparation of a medicament for treatment of a hepatocellular carcinoma, wherein said treatment further comprises lenvatinib.

Further provided is a use of lenvatinib in the preparation of a medicament for treatment of a hepatocellular carcinoma, wherein said treatment further comprises an EGFR inhibitor.

Further provided is a combination of lenvatinib and an EGFR inhibitor, for use in the treatment of hepatocellular carcinoma. Hepatocellular carcinoma is a preferred example of a carcinoma that would benefit from treatment with a combination of lenvatinib and an EGFR inhibitor.

A patient suffering from a carcinoma may be scored according to the Eastern Cooperative Oncology Group (ECOG) Performance Status, which is a scaled measure of general well-being where 0 is fully active and 5 is dead.

A carcinoma often is staged to provide information about the localization of the cancer; the cell type such as, adenocarcinoma or squamous cell carcinoma; the size of the cancer; whether the cancer has spread to nearby lymph nodes; whether the cancer has spread to a different part of the body; and the cancer grade, which refers to how abnormal the cancer cells look and how likely the tumor is to grow and spread.

A system that is often used for staging carcinomas is the TNM Staging System, in which the T refers to the size and extent of the main primary cancer; the N refers to the number of nearby lymph nodes to which cancer cells have spread; and the M refers to whether the cancer has metastasized.

However, for many cancers, the conventional staging systems have shown limitations. For example, several new systems have been proposed for hepatocellular carcinoma including the HCC Barcelona (BCLC) staging classification, which links the stage of the disease to a specific treatment strategy; and the Child-Pugh grade, which determines functionality of the liver buy measuring bilirubin levels in the blood, albumin levels in the blood, prothrombin time, presence of ascites and presence of an encephalopathy, whereby class A means the liver is working normally; class B means mild to moderate damage; and class C means there is severe liver damage.

Said carcinoma preferably expresses an EGF receptor selected from ERBB3 and EGFR (ERBB1), preferably EGFR. Said level of expression preferably is more than 2 times, more preferred more than 4 times the level of expression in non-carcinogenic cells of the same origin, preferably more than 2 times, more preferred more than 4 times the Normalized eXpression (NX) level of expression in non-carcinogenic cells of the same origin.

Said level of expression of an EGFR family member, preferably EGFR itself, can be determined at the RNA level, and/or at the protein level. As is known to a person skilled in the art, determination of a level of EGFR expression at the RNA level can be performed, for example, by Northern blotting, quantitative amplification reactions, for example reverse transcriptase quantitative polymerase chain reaction (rt-qPCR), array-based quantitative hybridization, serial analysis of gene expression (SAGE), and sequencing, especially next generation sequencing. Determination of a level of EGFR expression at the protein level can be performed, for example, by Western blotting, immunohistochemistry, enzyme-linked immunosorbent assays (ELISA), fluorescence-activated cell sorting (FACS), microfluidic immuno-sensors, and/or by coupling to EGFR-binding partners on beads or monolithic material, followed by quantification of the bound material.

Said level of expression of an EGF receptor, preferably EGFR, preferably is normalized, meaning that the level of expression of corrected for effects that arose from variations in the technology rather than from true differences in a level of EGF receptor expression between biological samples. As is known to a person skilled in the art, normalization may be performed by correcting for levels of expression of so-called household genes, and/or by determining the level of expression of an EGF receptor, preferably EGFR, as a ratio of a total number of analyzed gene expression products. For example, the expression level of an EGF receptor, preferably EGFR, mRNA can be determined by sequencing of a total of 1 million transcripts, and the level of expression of said EGF receptor, preferably EGFR, can be determined as relative expression in parts per million (ppm).

A patient suffering from a hepatocellular carcinoma may previously have been treated with a kinase inhibitor, such as a multi kinase inhibitor such as lenvatinib and/or sorafenib. For example, a patient suffering from a carcinoma such as hepatocellular carcinoma who previously has been treated with lenvatinib, but who was found unresponsive or became resistant during treatment, can successfully be treated with a combination of an EGFR inhibitor and lenvatinib according to the invention.

Lenvatinib preferably is provided orally at a daily dosage of 2-50 mg, such as about 8-24 mg, preferably 12-18 mg. Said daily dosage preferably is about 8 mg/day for a patient suffering from a carcinoma having a body weight of less than 60 kg, and 12 mg/day for a patient suffering from a carcinoma having a body weight of more than 60 kg.

Lenvatinib has multi-kinase inhibition activity with IC50 values for VEGFR1-3 kinases of 4.7 nmol/L, 3.0 nmol/L and 2.3 nmol/L, respectively (Capozzi et al., 2019. Cancer Management Res 11: 3847-3860). Other tyrosine-kinases are less sensitive to lenvatinib. For example, RET has an IC50 of 6.4 nmol/L, KIT has an IC50 of 85 nmol/L, fibroblast growth factor receptors (FGFR)1-4 have IC50 values of 61, 27, 52 and 43 nmol/L, respectively, and platelet-derived growth factor receptor PDGFR-α has an IC50 value of 29 nmol/L (Capozzi et al., 2019. Cancer Management Res 11: 3847-3860). Lenvatinib inhibits the VEGFR family with a Ki of 1 nmol/L, RET with a Ki of 1.5 nmol/L, FGFR1 with a Ki of 221 nmol/L, FGFR2 with a Ki of 8.21 nmol/L, and FGFR3 and KIT with Ki values of 151 nmol/L and 11 nmol/L, respectively (Capozzi et al., 2019. Cancer Management Res 11: 3847-3860).

Suitable EGFR inhibitors include gefitinib (N-(3-chloro-4-fluorophenyl)-7-methoxy-6-(3-morpholin-4-ylpropoxy)quinazolin-4-amine; preferably at a daily dosage of 50-500 mg, such as about 125-250 mg), erlotinib (N-(3-ethynylphenyl)-6,7-bis(2-methoxyethoxy)quinazolin-4-amine; preferably at a daily dosage of 50-500 mg, such as about 150 mg), lap atinib (N-[3-chloro-4-[(3-fluorophenyl) methoxy]phenyl]-6-[5-[(2-methylsulfonylethylamino)methyl]furan-2-yl]quinazolin-4-amine; preferably at a daily dosage of 500-2500 mg, such as about 1250 mg), cetuximab (recombinant epidermal growth factor receptor binding antibody fragment (FAB), preferably at a weekly dosage of 25-300 mg, preferably at about 100 mg; or at 100-500 mg/m2, preferably at 250-400 mg/m2), neratinib ((E)-N-[4-[3-chloro-4-(pyridin-2-ylmethoxy)anilino]-3-cyano-7-ethoxyquinolin-6-yl]-4-(dimethylamino)but-2-enamide; preferably at a daily dosage of 100-500 mg, such as about 240 mg), osimertinib (N-[2-[2-(dimethylamino)ethyl-methylamino]-4-methoxy-5-[[4-(1-methylindol-3-yl)pyrimidin-2-yl]amino]phenyl]prop-2-enamide; preferably at a daily dosage of 10-250 mg, such as about 80 mg), panitumumab (recombinant human IgG2 monoclonal antibody that binds specifically to the human epidermal growth factor receptor (EGFR), preferably at a weekly or biweekly dosage of 200-1000 mg, preferably at about 600 mg; or at 2-10 mg/kg, preferably at about 6 mg/kg), vandetanib (N-(4-bromo-2-fluorophenyl)-6-methoxy-7-[(1-methylpiperidin-4-yl)methoxy]quinazolin-4-amine; preferably at a daily dosage of 100-2500 mg, such as about 600 mg), necitumumab (recombinant monoclonal IgG1 antibody that prevents binding of EGF to its receptor; preferably at a weekly dosage of 200-2000 mg, preferably at about 800 mg), dacomitinib ((E)-N-[4-(3-chloro-4-fluoroanilino)-7-methoxyquinazolin-6-yl]-4-piperidin-1-ylbut-2-enamide; preferably at a daily dosage of 5-250 mg, such as about 45 mg), afatinib ((E)-N-[4-(3-chloro-4-fluoroanilino)-7-[(3S)-oxolan-3-yl]oxyquinazolin-6-yl]-4-(dimethylamino)but-2-enamide; preferably at a daily dosage of 5-250 mg, such as about 40 mg); canertinib (N-[4-(3-chloro-4-fluoroanilino)-7-(3-morpholin-4-ylpropoxy)quinazolin-6-yl]prop-2-enamide; preferably at a daily dosage of 5-1000 mg, such as about 100 mg), or a combination thereof.

Preferred EGFR inhibitors are gefitinib and erlotinib. Preferred combinations are lenvatinib and gefitinib, and lenvatinib and erlotinib.

A most preferred combination comprises lenvatinib and gefitinib. Said combination of lenvatinib and gefitinib as an Epidermal Growth Factor Receptor (EGFR) inhibitor for use in a method of treating a patient suffering from a hepatocellular carcinoma preferably comprises administration of a daily dosage of lenvatinib at about 8 mg/day for a patient suffering from a carcinoma having a body weight of less than 60 kg, and 12 mg/day for a patient suffering from a carcinoma having a body weight of more than 60 kg, combined with a daily half dose 0.125 g/day of gefitinib. If the combination is well tolerated, the daily dose of gefitinib may be adjusted to a complete dose of about 0.25 g/day, for example after one week.

In a combination for use according to the invention, lenvatinib is administrated simultaneously with, separately from, or sequentially to the EGF inhibitor. When administered as two distinct pharmaceutical preparations, they may be administered on the same day or on different days to a patient in need thereof, and using a similar or dissimilar administration protocol, e.g. daily, twice daily, biweekly, orally and/or by infusion. Said combination is preferably administered repeatedly according to a protocol that depends on the patient to be treated (age, weight, treatment history, etc.), which can be determined by a skilled physician. Said protocol may include daily administration for 1-30 days, such as 2 days, 10 days, or 21 days, followed by period of 1-14 days, such as 7 days, in which no inhibitor is administered.

Said combination of lenvatinib and an EGFR inhibitor for use in a method of treating a hepatocellular carcinoma according to the invention may further be combined with an immune checkpoint inhibitor such a PD1/PDL1 inhibitor and/or antibodies against CTLA-4.

Suitable immune checkpoint inhibitors are PD1/PDL1 inhibitors such as antibodies, including pembrolizumab (Merck), nivolumab (Bristol-Myers Squibb), pidilizumab (Medivation/Pfizer), MEDI0680 (AMP-514; AstraZeneca) and PDR001 (Novartis); fusion proteins such as a PD-L2 Fc fusion protein (AMP-224; GlaxoSmithKline); atezolizumab (Roche/Genentech), avelumab (Merck/Serono and Pfizer), durvalumab (AstraZeneca), BMS-936559 (Bristol-Myers Squibb); and small molecule inhibitors such as PD-1/PD-L1 Inhibitor 1 (WO2015034820; (2S)-1-[[2,6-dimethoxy-4-[(2-methyl-3-phenylphenyl)methoxy]phenyl]methyl]piperidine-2-carboxylic acid), BMS202 (PD-1/PD-L1 Inhibitor 2; WO2015034820; N-[2-[[[2-methoxy-6-[(2-methyl[1,1′-biphenyl]-3-yl)methoxy]-3-pyridinyl]methyl]amino]ethyl]-acetamide), and PD-1/PD-L1 Inhibitor 3 (WO/2014/151634; (3S,6S,12S,15S,18S,21S,24S,27S,30R,39S,42S,47aS)-3-((1H-imidazol-5-yl)methyl)-12,18-bis((1H-indol-3-yl)methyl)-N,42-bis(2-amino-2-oxoethyl)-36-benzyl-21,24-dibutyl-27-(3-guanidinopropyl)-15-(hydroxymethyl)-6-isobutyl-8,20,23,38,39-pentamethyl-1,4,7,10,13). Further anti-PD1 molecules include ladiratuzumab vedotin (Seattle Genetics).

Said combination of lenvatinib and an EGFR inhibitor for use in a method of treating a hepatocellular carcinoma according to the invention may further be combined with a fibroblast growth factor inhibitor, including AZD4547 (N-[5-[2-(3,5-dimethoxyphenyl)ethyl]-1H-pyrazol-3-yl]-4-[(3S,5R)-3,5-dimethylpiperazin-1-yl]benzamide; preferably at a daily or twice daily orally dosage of 10-250 mg, such as about 80 mg); Ly2874455 (2-{4-[(E)-2-{5-[(1R)-1-(3,5-Dichloro-4-pyridinyl)ethoxy]-1H-indazol-3-yl}vinyl]-1H-pyrazol-1-yl}ethanol; preferably at a daily or twice daily orally dosage of 1-100 mg, such as about 8-24 mg); CH5183284 (Debio-1347; [5-amino-1-(2-methyl-3H-benzimidazol-5-yl)pyrazol-4-yl]-(1H-indol-2-yl)methanone; preferably at a daily or twice daily orally dosage of 5-1000 mg, such as about 10-150 mg); infigratinib (BGJ398; 3-(2,6-dichloro-3,5-dimethoxyphenyl)-1-[6-[4-(4-ethylpiperazin-1-yl)anilino]pyrimidin-4-yl]-1-methylurea; preferably at a daily or twice daily orally dosage of 50-10000 mg, such as about 150 mg); pemigatinib (INCB054828; 11-(2,6-difluoro-3,5-dimethoxyphenyl)-13-ethyl-4-(morpholin-4-ylmethyl)-5,7,11,13-tetrazatricyclo[7.4.0.02,6]trideca-1,3,6,8-tetraen-12-one; preferably at a daily or twice daily orally dosage of 1-100 mg, such as about 13.5 mg); rogaratinib (4-[[4-amino-6-(methoxymethyl)-5-(7-methoxy-5-methyl-1-benzothiophen-2-yl)pyrrolo[2,1-f][1,2,4]triazin-7-yl]methyl]piperazin-2-one; preferably at a daily or twice daily orally dosage of 10-1500 mg, such as about 800 mg); PRN1371 (8-[3-(4-Acryloyl-1-piperazinyl)propyl]-6-(2,6-dichloro-3,5-dimethoxyphenyl)-2-(methylamino)pyrido[2,3-d]pyrimidin-7(8H)-one; preferably at a daily or twice daily orally dosage of 1-150 mg, such as about 35 mg); futibatinib (TAS-120; 1-[(3S)-3-[4-amino-3-[2-(3,5-dimethoxyphenyl)ethynyl]pyrazolo[3,4-d]pyrimidin-1-yl]pyrrolidin-1-yl]prop-2-en-1-one; preferably at a daily or twice daily orally dosage of 1-200 mg, such as about 4-24 mg); fisogatinib (BLU-554; N-[(3S,4S)-3-[[6-(2,6-dichloro-3,5-dimethoxyphenyl)quinazolin-2-yl]amino]oxan-4-yl]prop-2-enamide; preferably at a daily or twice daily orally dosage of 10-600 mg, such as about 200 mg); H3B-6527 (N-{2-[(6-{[(2,6-dichloro-3,5-dimethoxyphenyl)carbamoyl](methyl)amino}-4-pyrimidinyl)amino]-5-(4-ethyl-1-piperazinyl)phenyl}acrylamide; preferably at a daily or twice daily orally dosage of 100-2000 mg, such as about 500-1000 mg); or a combination thereof.

A combination of lenvatinib and an EGFR inhibitor for use in a method of treating a hepatocellular carcinoma may, in addition, be combined with one or more chemotherapeutical agents, including cytotoxic agents, immunomodulating agents and immunotoxic agents.

Said one or more chemotherapeutical agents include alkylating agents such as busulfan, melphalan, carboplatin, cisplatin, cyclophosphamide, dacarbazine, carmustine, nimustin, lomustine, ifosfamide, temozolomide, navelbine and altretamine, antibiotics such as leomycin, doxorubicin, adriamycin, idarubicin, epirubicin and plicamycin, antimetabolites such as sulfonamides, folic acid antagonists, gemcitabine, 5-fluorouracil (5-FU), leucovorine, leucovorine with 5-FU, 5-FU with calcium folinate and leucovorin, capecitabine, mercaptopurine, cladribine, pentostatine, methotrexate, raltitrexed, pemetrexed, thioguanine, and camptothecin derivates such as topotecan and irinotecan, hormones and antagonists thereof such as flutamide, goserelin, mitotane and tamoxifen, mustard gas derivatives such as melphalan, carmustine and nitrogen mustard, and alkaloids such as taxanes, docetaxel, paclitaxel, etoposide, vincristine, vinblastine and vinorelbine.

Said combination of lenvatinib and an EGFR inhibitor for use according to the invention may be combined or alternated with a G2 checkpoint abrogator such as SB-218078 (9,10,11,12-tetrahydro-9,12-epoxy-1H-diindolo[1,2,3-fg:3′,2′,1′-kl]pyrrolo[3,4-i][1,6]benzodiazocine-1,3(2H)-dione) and UCN-01 ((3R,9S,10R,11R,13R)-2,3,10,11,12,13-hexahydro-3-hydroxy-10-methoxy-9-methyl-11-(methylamino)-9,13-epoxy-1H,9H-diindolo[1,2,3-gh:3′,2′,1′-lm]pyrrolo[3,4-j][1,7]benzodiazonin-1-one); a focal adhesion kinase inhibitor such as amlodipine besylate (benzenesulfonic acid; 3-O-ethyl 5-O-methyl 2-(2-aminoethoxymethyl)-4-(2-chlorophenyl)-6-methyl-1,4-dihydropyridine-3,5-dicarboxylate), defactinib (N-methyl-4-[[4-[[3-[methyl(methylsulfonyl)amino]pyrazin-2-yl]methylamino]-5-(trifluoromethyl)pyrimidin-2-yl]amino]benzamide), or GSK2256098 (2-[[5-chloro-2-[(5-methyl-2-propan-2-ylpyrazol-3-yl)amino]pyridin-4-yl]amino]-N-methoxybenzamide); and/or an antimetabolite such as sulfonamides, folic acid antagonists, gemcitabine, 5-fluorouracil (5-FU), leucovorine, leucovorine with 5-FU, 5-FU with capecitabine, mercaptopurine, cladribine, pentostatine, methotrexate, raltitrexed, pemetrexed, thioguanine, and camptothecin derivates such as topotecan and irinotecan.

The invention further provides a method of treating a hepatocellular carcinoma in a subject, the method comprising the simultaneous, separate or sequential administering to the subject of lenvatinib and an EGFR inhibitor, whether or not combined with an immune checkpoint inhibitor, a FGF inhibitor, a chemotherapeutical agent, or a combination thereof.

### 4.3 Compositions

A combination of lenvatinib and an EGFR inhibitor for use according to the invention may be provided in one pharmaceutical preparation, or as two or more distinct pharmaceutical preparations. Said single or distinct pharmaceutical preparations further comprise pharmaceutically acceptable excipients, as is known to a person skilled in the art. For oral administration, a preferred pharmaceutical preparation is provided by a tablet. The term “tablet” encompasses a “capsule” and a “caplet”.

Pharmaceutically acceptable excipients include diluents, binders or granulating ingredients, a carbohydrate such as starch, a starch derivative such as starch acetate and/or maltodextrin, a polyol such as xylitol, sorbitol and/or mannitol, lactose such as α-lactose monohydrate, anhydrous α-lactose, anhydrous ß-lactose, spray-dried lactose, and/or agglomerated lactose, a sugar such as dextrose, maltose, dextrate and/or inulin, or combinations thereof, glidants (flow aids) and lubricants to ensure efficient tableting, and sweeteners or flavors to enhance taste.

The invention therefore provides a pharmaceutical composition, comprising lenvatinib and an EGFR inhibitor. Said pharmaceutical composition preferably is for use in a method of treating a patient suffering from a hepatocellular carcinoma.

The invention further provides a kit of parts, comprising lenvatinib and an EGFR inhibitor, as a combined preparation for simultaneous, separate or sequential use in the treatment of a hepatocellular carcinoma in a subject. Said kit of parts may further comprise a pharmaceutical composition comprising a suitable immune checkpoint inhibitor such as a PD1/PDL1 inhibitor. Said kit of parts may further comprise a pharmaceutical composition comprising an immune checkpoint inhibitor, a FGF inhibitor, a chemotherapeutical agent, or a combination thereof.

For the purpose of clarity and a concise description, features are described herein as part of the same or separate aspects and preferred embodiments thereof, however, it will be appreciated that the scope of the invention may include embodiments having combinations of all or some of the features described.

The invention will now be illustrated by the following examples, which are provided by way of illustration and not of limitation and it will be understood that many variations in the methods described and the amounts indicated can be made without departing from the spirit of the invention and the scope of the appended claims.

### 5 EXAMPLES

### Example 1

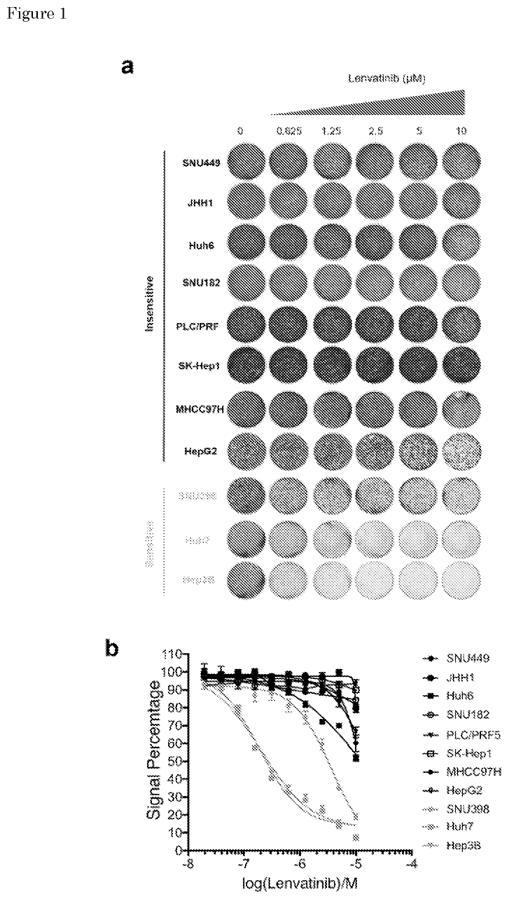

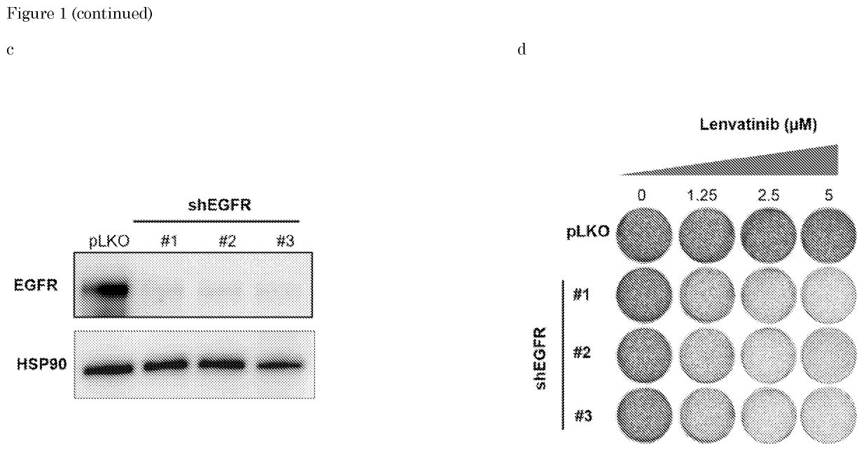

To study how liver cancer cell lines respond to lenvatinib in vitro, we determined the efficacy of lenvatinib in 11 liver cancer cell lines using a long-term proliferation assay and short-term CellTiter-Blue viability assays. The results show that most cell lines are not insensitive to lenvatinib in vitro (FIG. 1a, b).

To find potential drug targets that can act synergistically with lenvatinib in HCC, we performed a synthetic lethality screen with lenvatinib using CRISPR-Cas9 genetic screen targeting the human kinome in the HCC cell line SNU449. SNU449 cells were infected with the lentiviral kinome gRNA library and cultured in the absence or presence of lenvatinib for 14 days. After this, genomic DNA was isolated from both treated and untreated cells, and the relative abundance of gRNA was determined by next generation sequencing of the bar code contained in each gRNA vector in three biological replicates as described (Evers et al., 2016. Nat Biotechnol 34: 631-3). Several independent gRNA vectors targeting EGFR were identified as being depleted specifically in the presence of lenvatinib, suggesting that EGFR inhibition is synthetic lethal with lenvatinib. To validate this finding, we infected SNU449 cells with three EGFR shRNA vectors (all of which reduced EGFR levels; FIG. 1c) and cultured these cells with or without lenvatinib for about 10 days. The results showed that suppression of EGFR in combination with lenvatinib caused a marked inhibition of proliferation in SNU449 cells (FIG. 1d).

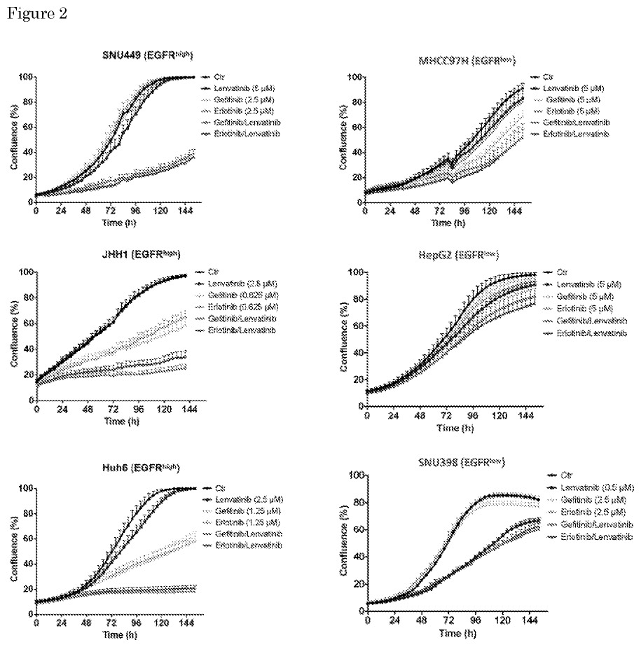

Subsequently, liver cancer cell lines, which expressed relatively high levels of EGFR (SNU449, JHH1, and Huh6) or low levels of EGFR (MHCC97H, SNU398, and HepG2), were treated with a combination of lenvatinib and EGFR inhibitors (gefitinib and erlotinib). The results of IncuCyte® short-term cell proliferation assays showed that strong synergy between these two types of inhibitors was observed in liver cancer cell lines with high level of EGFR, but not in liver cancer cell lines with low levels of EGFR (FIG. 2). Comparable results were observed in long-term colony formation assays (data not shown).

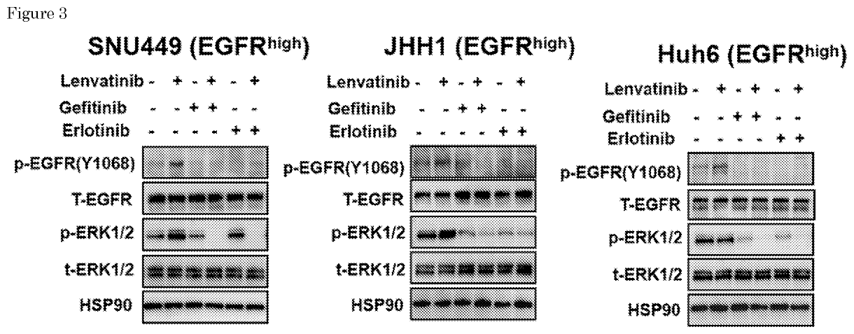

To address the molecular mechanism underlying the synergy between lenvatinib and EGFR inhibition in liver cancer, we tested lysates of drug-treated cells with phospho-protein-specific antibodies that identify the activated state of components of the EGFR signaling pathway. We observed that treatment of all three EGFR-high liver cancer cell lines with lenvatinib resulted in a strong increase in Tyr 1068 phosphorylation of EGFR, which reflects activation of the receptor, and phosphorylation of its downstream kinase ERK1/2 (FIG. 3). This observation suggests that a powerful feedback activation of EGFR is elicited by lenvatinib treatment. Moreover, co-treatment of these cells with a combination of lenvatinib and either gefitinib or erlotinib prevented this feedback activation of EGFR, as well as activation of ERK1/2 (FIG. 3). GSEA analyses following RNA-sequencing of lenvatinib, gefitinib, or the two-drug combination treatment further indicated additional gene set enrichments related to downregulation of EGFR signaling after combination treatment (data not shown). Consistently, results of real-time PCR showed that the combination treatment dramatically decreased expression levels of 10 conserved transcriptional targets of MAPK/ERK signaling (PHLDA1, SPRY2, SPRY4, DUSP4, DUSP6, CCND1, EPHA2, EPHA4, ETV4, and ETV5), indicating that the combination treatment can block MAPK pathway activity in these liver cancer cells (data not shown).

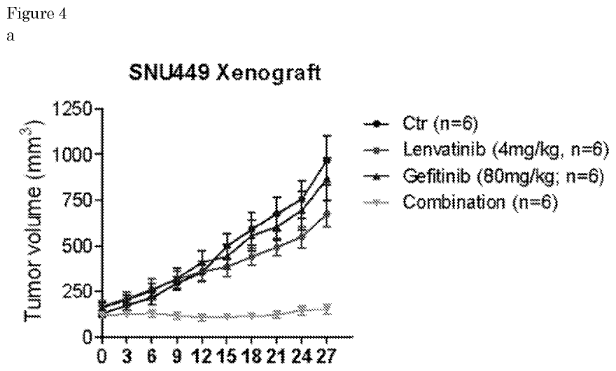

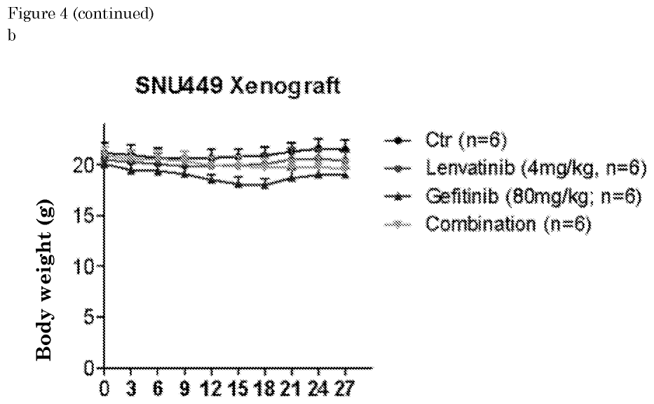

To assess whether the in vitro findings can be recapitulated in vivo, SNU449 (high EGFR level) cells were injected into nude mice. Upon tumor establishment, xenografts were treated with vehicle, lenvatinib, gefitinib, or the combination for about 27 days. Our results show that the combination of lenvatinib and gefitinib elicited a complete growth inhibition of SNU449 cells without any toxicity, while single drug treatment showed little antitumor effect in vivo (FIG. 4a,b). To study the expression level of EGFR in primary liver cancer tumors, we analyzed EGFR using a tissue microarray (TMA) containing 298 HCC specimens by immunochemical analysis. Levels of EGFR in tumor tissues were either classified as negative, low, moderate, or high expression. About 52.6% of total (157 cases) were identified as EGFR-positive HCC patients when assessed by H-score with a magnification rule (FIG. 5). These results demonstrated that more than 50% HCC patients might benefit from the combination of lenvatinib and EGFR inhibitors in clinic.

### Example 2

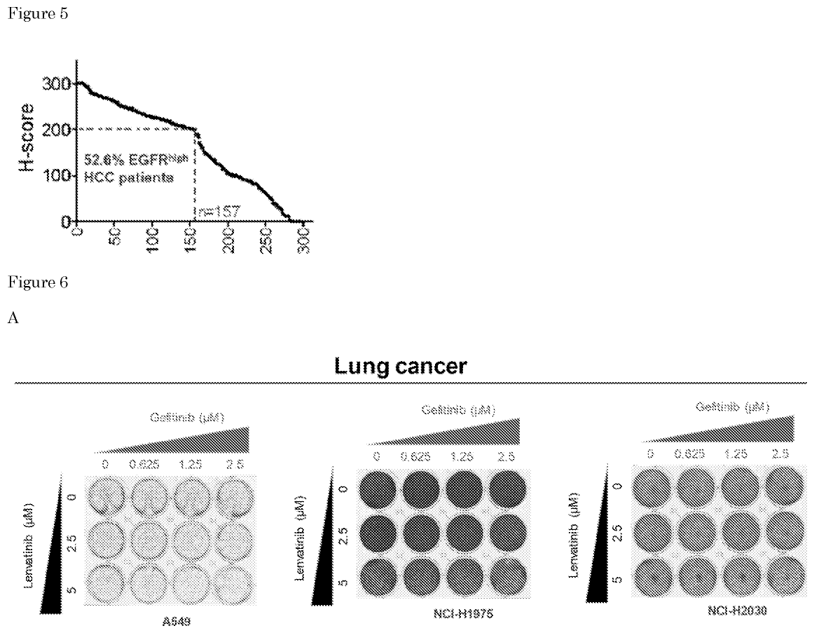

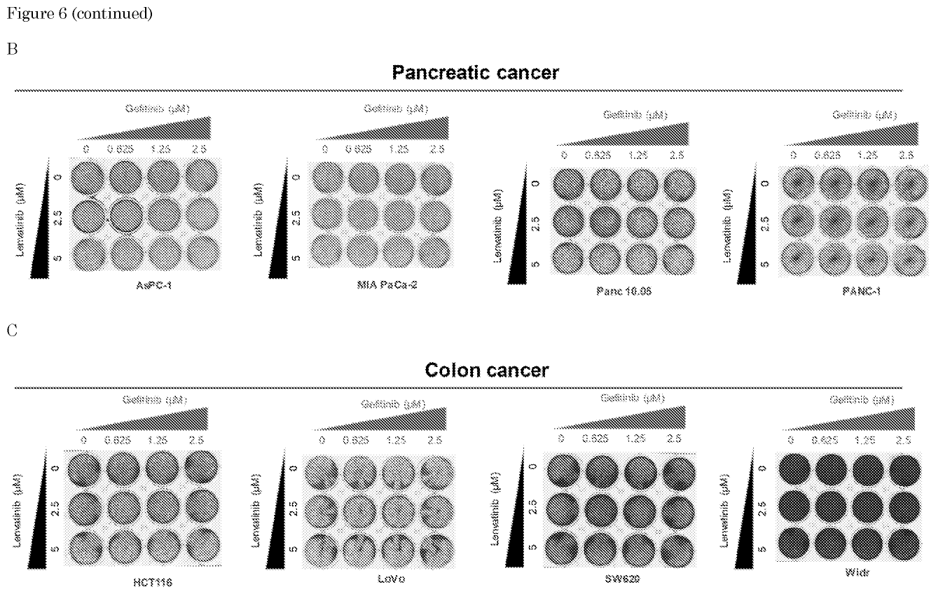

Distinct lung cancer cells (A549, NCI-H1975 and NCI-H2030), pancreatic cancer cells (AsPC-1, MIA PaCa-2, Panc 10.05 and PANC-1), and colon cancer cells HCT116, LoVo, SW620 and WiDr) were grown and treated with lenvatinib, the EGFR inhibitor gefitinib, or the combination of lenvatinib and the EGFR inhibitor gefitinib. Concentrations used were 0, 0.625, 1.25 and 2.5 microM of gefitinib, and 0, 2.5 and 5 microM of lenvatinib. Cells were fixed and stained after 10-14 days. All cell lines are available from the American Type Culture Collection at ATCC.org. As is shown in FIG. 6, a combination of lenvatinib and the EGFR inhibitor gefitinib did not reduce growth of these carcinoma cells.

1. A method of treating hepatocellular carcinoma in a subject comprising administration of a combination of lenvatinib and an Epidermal Growth Factor Receptor (EGFR) inhibitor.

2. The method according to claim 1, wherein the hepatocellular carcinoma has high EGFR expression levels of >200, as determined with the El-score method.

3. The method according to claim 1, wherein said EGFR inhibitor is an EGFR tyrosine kinase inhibitor (TKI), or a binding molecule that prevents binding of a ligand to the EGFR.

4. The method according to claim 1, wherein said EGFR inhibitor comprises gefitinib, erlotinib, lapatinib, cetuximab, neratinib, osimertinib, panitumumab, vandetanib, necitumumab, dacomitinib, afatinib, canertinib, or a combination thereof.

5. The method according to claim 1, wherein said subject patient has previously been treated with an inhibitor of multiple receptor tyrosine kinases.

6. The method according to claim 1, wherein said subject patient has previously been treated with lenvatinib and/or sorafenib.

7. The method according to claim 1, wherein said subject has become resistant to said one or more inhibitors of multiple receptor tyrosine kinases.

8. The method according to claim 1, wherein the administration of a combination of lenvatinib and an EGFR inhibitor is combined or alternated with the administration of an immune checkpoint inhibitor.

9. The method according to claim 1, wherein the administration of a combination of lenvatinib and an EGFR inhibitor is combined or alternated with the administration of a PD1/PDL1 inhibitor.

10. A pharmaceutical preparation, comprising lenvatinib and an Epidermal Growth Factor Receptor (EGFR) inhibitor.

11. The pharmaceutical preparation according to claim 10, comprising a pharmaceutical preparation comprising lenvatinib and a pharmaceutical preparation comprising an EGER inhibitor.

12. A method of treating hepatocellular carcinoma in a subject comprising administrating the pharmaceutical preparation according to claim 10 to the subject.

13. A kit of parts, comprising lenvatinib and an EGFR inhibitor as a combined preparation for simultaneous, separate or sequential use in the treatment of a hepatocellular carcinoma in a subject.

14. The method of claim 12 comprising simultaneous, separate or sequential administration of lenvatinib and an EGFR inhibitor to the subject.

15. The method of claim 14, further comprising administering an immune checkpoint inhibitor, preferably a PD1/PDL1 inhibitor to the subject.
